(19) United States
(12) Patent Application Publication
(10) Pub. No.: US 2012/0265068 A1
(43) Pub. Date: Oct. 18, 2012

(54) METHOD AND APPARATUS FOR DISPLAYING THERMAL RISK INDICATOR

(75) Inventors: Kurt SANDSTROM, Gangwon-do (KR); Dae-young KIM, Gangwon-do (KR); Tae-yun KIM, Gangwon-do (KR); You-chun PU, Gangwon-do (KR); Sung-in CHO, Gangwon-do (KR)

(73) Assignee: SAMSUNG MEDISON CO., LTD.

(21) Appl. No.: 13/446,814

(22) Filed: Apr. 13, 2012

(30) Foreign Application Priority Data
Apr. 14, 2011 (KR) 10-2011-0034514

(51) Int. Cl. A61B 8/00

(52) U.S. Cl. 600/437

(57) ABSTRACT

An apparatus for displaying a thermal risk indicator, which measures at least one analyzing indicator about a risk of a thermal bio-effect by analyzing an ultrasound beam generated from an ultrasound output unit of a transmitting transducer, calculates a thermal risk indicator based on the at least one analyzing indicator, and displays the thermal risk indicator through a display unit.

### CROSS-REFERENCE TO RELATED PATENT APPLICATION

This application claims the benefit of Korean Patent Application No. 10-2011-0034514, filed on Apr. 14, 2011, in the Korean Intellectual Property Office, the disclosure of which is incorporated herein in its entirety by reference.

### BACKGROUND OF THE INVENTION

1. Field of the Invention

The present invention relates to a method and apparatus for displaying a thermal risk indicator, and more particularly, to a method and apparatus for displaying a thermal risk indicator, in which the thermal risk indicator is calculated by using at least one analyzing indicator about a thermal bio-effect.

2. Description of the Related Art

Ultrasound diagnosis apparatuses transmit ultrasound signals from a surface of a subject, e.g, a human body or an animal body, towards a predetermined part within the body (i.e., an object such as a fetus or an internal organ) and use information about ultrasound signals reflected from a tissue within the body to obtain images related to a cross-section of soft tissue or blood flow.

Due to its compact and cheap design, real-time display, and high stability with no risk of exposure to X-rays or other radiations, such ultrasound diagnosis apparatuses have been widely used together with other image diagnostic devices such as X-ray diagnostic devices, computerized tomography (CT) scanners, magnetic resonance imaging (MRI) devices, and nuclear medicine diagnostic devices.

In general, an ultrasound diagnosis apparatus transmits an ultrasound signal to a target and receives an ultrasound signal (i.e., an ultrasound echo signal) reflected from the target to form a two-dimensional (2D) or three-dimensional (3D) ultrasound image of the target. The ultrasound diagnosis apparatus outputs the 2D or 3D ultrasound image through an output unit.

It is important for an ultrasound diagnosis apparatus to output an accurate ultrasound image in consideration of various analyzing indicators according to a tissue or position to which an ultrasound beam is irradiated.

### SUMMARY OF THE INVENTION

The present invention provides a thermal risk indicator for monitoring and controlling a thermal risk that is generated when a biological tissue absorbs energy of an ultrasound beam, which is provided to a user using an ultrasound diagnosis apparatus.

The present invention also provides exponential rise data of a thermal bio-effect according to a linear temperature rise of a biological tissue, which is provided to the user.

The present invention also provides linear rise data of a thermal bio-effect according to time when an ultrasound beam is irradiated to a biological tissue of a signal region, which is provided to the user.

According to an aspect of the present invention, there is provided an apparatus for displaying a thermal risk indicator, the apparatus including a measuring unit for measuring at least one analyzing indicator about a risk of a thermal bio-effect by analyzing an ultrasound beam generated from an ultrasound output unit of a transmitting transducer; a calculating unit for calculating a thermal risk indicator based on the at least one analyzing indicator; and a display unit for displaying the thermal risk indicator.

The at least one analyzing indicator may include an estimated value ΔT(t) by which a temperature increases hourly, a possible maximum temperature rise range value ΔT_{MAX} that is calculated with respect to a preset operating condition, an elapsed time ‘t’ after the operating condition is reset, or a stable used time t_{SU} for a tissue and position to which the ultrasound beam is irradiated.

The possible maximum temperature rise range value ΔT_{MAX} is a maximum value in a burst mode including an acoustic radiation force impulse (ARFI).

When an ultrasound beam is used as a multi-pulse, if a temperature does not correspond to a thermal equilibrium condition in a state where a pulse of an ultrasound beam is off, the possible maximum temperature rise range value ΔT_{MAX} may include a value that is calculated at a point of time when the last pulse is terminated.

The apparatus may further include a determination unit for determining whether the ‘t’ reaches the stable used time T_{SU}.

The determination unit may automatically reduce or shut off a transmission voltage according to a determination result obtained by the determination unit.

The determination unit may display an alarm message or information data according to a determination result obtained by the determination unit.

According to another aspect of the present invention, there is provided a method of displaying a thermal risk indicator, the method including measuring at least one analyzing indicator about a risk of a thermal bio-effect by analyzing an ultrasound beam generated from an ultrasound output unit of a transmitting transducer; calculating a thermal risk indicator based on the at least one analyzing indicator; and displaying the thermal risk indicator.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram of a thermal risk indicator displaying apparatus according to an embodiment of the present invention;

FIGS. 2 and 3 are diagrams for showing cases where a thermal risk indicator is displayed, according to embodiments of the present invention;

FIG. 4 is a graph for showing a thermal change according to time in a burst mode, according to an embodiment of the present invention;

FIG. 5 is a flowchart of a method of displaying a thermal risk indicator, according to an embodiment of the present invention.

### DETAILED DESCRIPTION OF THE INVENTION

Reference will now be made in detail to embodiments, examples of which are illustrated in the accompanying drawings. Also, while describing the embodiments, detailed descriptions about related well-known functions or configurations that may diminish the clarity of the points of the embodiments of the present invention are omitted. Terms or words used herein shall not be limited to their common or dictionary meanings, and have meanings corresponding to technical aspects of the embodiments of the present invention so as to most suitably express the embodiments of the present invention. Expressions such as “at least one of,” when preceding a list of elements, modify the entire list of elements and do not modify the individual elements of the list.

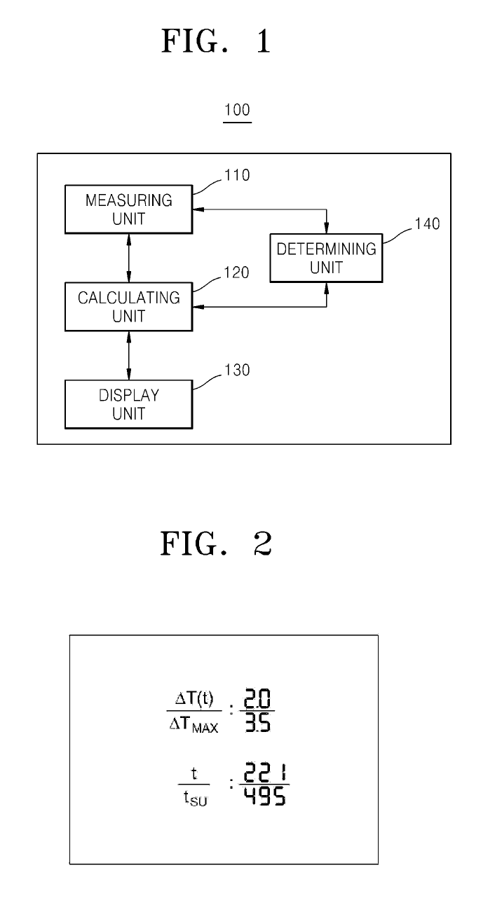

FIG. 1 is a block diagram of a thermal risk indicator displaying apparatus 100 according to an embodiment of the present invention.

Referring to FIG. 1, the thermal risk indicator displaying apparatus 100 includes a measuring unit 110, a calculating unit 120, and a display unit 130.

The measuring unit 110 measures at least one analyzing indicator about a risk of a thermal bio-effect by analyzing an ultrasound beam generated from an ultrasound output unit of a transmitting transducer.

The calculating unit 120 calculates a thermal risk indicator based on the measured at least one analyzing indicator.

The display unit 130 displays the thermal risk indicator.

According to the present embodiment, the at least one analyzing indicator may include at least one of an estimated value ΔT(t) by which a temperature increases hourly, a possible maximum temperature rise range value ΔT_{MAX} that is calculated with respect to a preset operating condition, an elapsed time ‘t’ after the operating condition is reset, and a stable used time T_{SU} in relation to a tissue and position to which the ultrasound beam is irradiated.

According to the present embodiment, the possible maximum temperature rise range value ΔT_{MAX} may be a maximum value in burst modes including an acoustic radiation force impulse (ARFI).

According to the present embodiment, when an ultrasound beam is used as a multi-pulse, if a temperature does not correspond to a thermal equilibrium condition in a state where a pulse of the ultrasound beam is off, the possible maximum temperature rise range value ΔT_{MAX} may include a value that is calculated at a point of time when the last pulse is terminated.

According to the present embodiment, when the ultrasound beams is used as a multi-pulse, the possible maximum temperature rise range value ΔT_{MAX} may include a value that is calculated at a point of time when an on-state of the burst mode is terminated.

According to the present embodiment, a tissue to which the ultrasound beam is irradiated may include at least one of a soft tissue, a bone, and the like.

According to the present embodiment, the thermal risk indicator may be calculated according to Formula 1 or 2 below:

$$\begin{matrix}\frac{Δ T ( t )}{Δ {T}_{MAX}} & ( 1 ) \\ \frac{t}{{t}_{SU}} & ( 2 )\end{matrix}$$

According to the present embodiment, the thermal risk indicator obtained according to Formula 1 or 2 may have a ratio between analyzing indicators of Formulas 1 and 2.

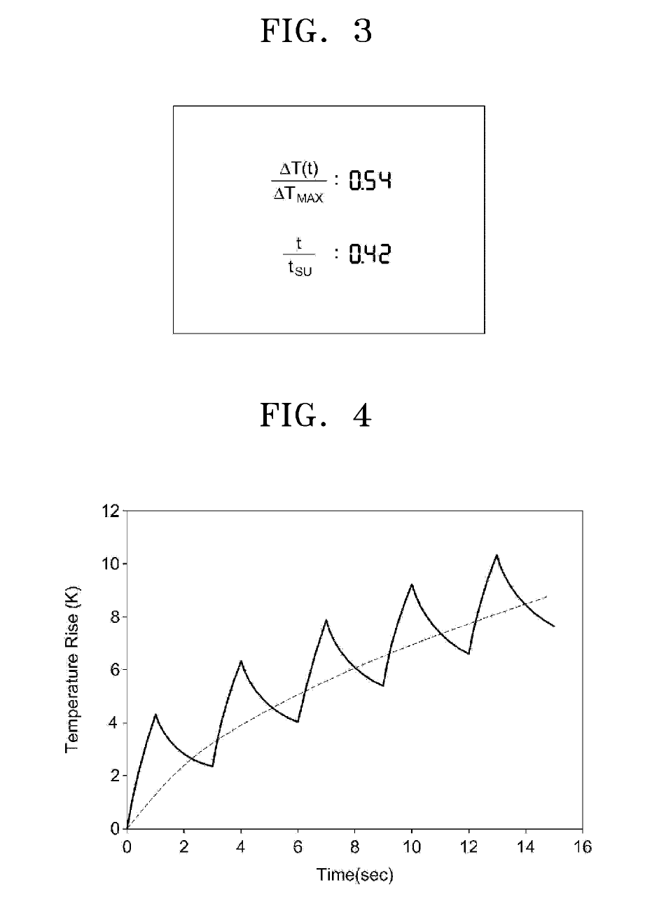

FIGS. 2 and 3 are diagrams for showing cases where a thermal risk indicator is displayed, according to embodiments of the present invention.

For example, if it is assumed that the estimated value ΔT(t) is 1.9, the possible maximum temperature rise range value ΔT_{MAX} is 3.5, the elapsed time ‘t’ is 210 seconds, and the stable used time t_{SU} is 495, the thermal risk indicators calculated according to Formulae 1 and 2 may provide ratios to a user, as shown in FIG. 3.

As another example, when the ultrasound beam is irradiated to the tissue for 11 seconds, a temperature increases as time passes, the elapsed time ‘t’ is 221, and the estimated value ΔT(t) is 2.0. Thus, ratio data displayed as the thermal risk indicator changes, as shown in FIG. 2.

According to the present embodiment, the display unit 130 may display each of the thermal risk indicators that are respectively calculated according to Formulas 1 and 2.

According to the present embodiment, the display unit 130 may emphasize and display the thermal risk indicator calculated according to Formula 1 when the estimated value ΔT(t) reaches the possible maximum temperature rise range value ΔT_{MAX}.

In this case, the display unit 130 may emphasize and display the thermal risk indicator calculated according to Formula 1 by using at least one of bold lettering, colored letters, and flickering letters.

According to the present embodiment, the display unit 130 may emphasize and display the thermal risk indicator calculated according to Formula 2 when the elapsed time ‘t’ reaches the stable used time t_{SU}.

In this case, the display unit 130 may emphasize and display the thermal risk indicator calculated according to Formula 2 by using at least one of bold lettering, colored letters, and flickering letters.

The thermal risk indicator displaying apparatus 100 may include a determining unit 140 for determining whether the elapsed time ‘t’ reaches the stable used time t_{SU}. A transmission voltage may be automatically reduced or shut off according to a result of the determining by the determining unit 140.

According to the present embodiment, the determining unit 140 may display an alarm message or information data according to the determination result.

When a position of a biological tissue to which the ultrasound beam is irradiated is not changed and an operational state of a system for irradiating the ultrasound beam is changed, the thermal risk indicator displaying apparatus 100 may update the estimated value ΔT(t), the possible maximum temperature rise range value ΔT_{MAX}, and the stable used time T_{SU}, and the elapsed time ‘t’ continues to elapse.

When the operational state of the system for irradiating the ultrasound beam is maintained and the type of a biological tissue to which the ultrasound beam is irradiated is not changed, and a position to which the ultrasound beam is irradiated is changed, the thermal risk indicator displaying apparatus 100 may maintain the possible maximum temperature rise range value ΔT_{MAX} and the stable used time t_{SU}, and may reset the estimated value ΔT(t) and the elapsed time ‘t’ to 0.

When the operational state of the system for irradiating the ultrasound beam is changed or the type of the biological tissue to which the ultrasound beam is irradiated is changed, the thermal risk indicator displaying apparatus 100 may calculate the possible maximum temperature rise range value ΔT_{MAX} and the stable used time t_{SU} again and may reset the estimated value ΔT(t) and the elapsed time ‘t’ to 0.

FIG. 4 is a graph for showing a thermal change according to time in a burst mode, according to an embodiment of the present invention.

According to the present embodiment, as shown in FIG. 4, the display unit 130 may display the thermal risk indicator as a standard image mode in which a temperature of a normal state of a biological tissue increases.

According to the present embodiment, as shown in FIG. 4, the display unit 130 may display the thermal risk indicator in a burst mode for an acoustic radiation force image, or the like, in which a temperature of a biological tissue changes remarkably.

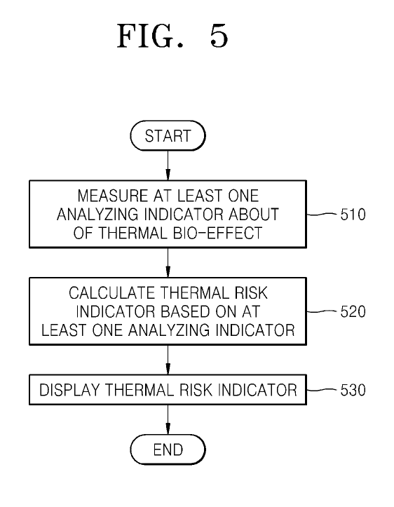

FIG. 5 is a flowchart of a method of displaying a thermal risk indicator, according to an embodiment of the present invention.

Referring to FIG. 5, the thermal risk indicator displaying apparatus 100 may measure at least one analyzing indicator about a risk of a thermal bio-effect by analyzing an ultrasound beam generated from an ultrasound output unit of a transmitting transducer (operation 510).

According to the present embodiment, the thermal risk indicator displaying apparatus 100 calculates a thermal risk indicator based on the measured at least one analyzing indicator (operation 520).

The thermal risk indicator displaying apparatus 100 displays the thermal risk indicator (operation 530).

Embodiments of the present invention may include a computer readable medium including program commands for executing operations implemented through various computers. The computer readable medium can store program commands, data files, data structures or combinations thereof. The program commands recorded in the medium may be specially designed and configured for the present invention or be known to those of ordinary skill in the field of computer software. Examples of a computer readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands. Examples of the program commands include a machine language code created by a compiler and a high-level language code executable by a computer using an interpreter and the like. The aforementioned hardware devices may include one or more software modules in order to execute operations of the present invention, or vice versa.

According to one or more embodiments of the present invention, a thermal risk indicator for monitoring and controlling a thermal risk that is generated from a biological tissue absorbing energy of an ultrasound beam may be provided to a user using an ultrasound diagnosis apparatus.

According to one or more embodiments of the present invention, exponential rise data of a thermal bio-effect according to a linear temperature rise of a biological tissue may be provided to the user of the ultrasound diagnosis apparatus.

According to one or more embodiments of the present invention, linear rise data of a thermal bio-effect according to time when an ultrasound beam is irradiated to a biological tissue of a signal region may be provided to the user of the ultrasound diagnosis apparatus.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An apparatus for displaying a thermal risk indicator, the apparatus comprising:
a measuring unit for measuring at least one analyzing indicator about a risk of a thermal bio-effect by analyzing an ultrasound beam generated from an ultrasound output unit of a transmitting transducer;
a calculating unit for calculating a thermal risk indicator based on the at least one analyzing indicator; and
a display unit for displaying the thermal risk indicator.

2. The apparatus of claim 1, wherein the at least one analyzing indicator comprises an estimated value ΔT(t) by which a temperature increases hourly, a possible maximum temperature rise range value ΔT_{MAX} that is calculated with respect to a preset operating condition, an elapsed time ‘t’ after the operating condition is reset, or a stable used time t_{SU} for a tissue and position to which the ultrasound beam is irradiated.

3. The apparatus of claim 2, wherein the possible maximum temperature rise range value ΔT_{MAX} is a value that is obtained by calculating the thermal risk indicator in burst modes comprising an acoustic radiation force impulse (ARFI).

4. The apparatus of claim 3, wherein the possible maximum temperature rise range value ΔT_{MAX} comprises a value that is calculated at a point of time when an on-state of the burst mode is terminated, when the ultrasound beam is used as a multi-pulse.

5. The apparatus of claim 2, wherein a tissue to which the ultrasound beam is irradiated comprises at least one of a soft tissue and a bone.

6. The apparatus of claim 2, wherein the thermal risk indicator is calculated according to Formula 1 or Formula 2 below:
$$\begin{matrix}\frac{Δ T ( t )}{Δ {T}_{MAX}} & ( 1 ) \\ \frac{t}{{t}_{SU}} & ( 2 )\end{matrix}$$

7. The apparatus of claim 6, wherein the thermal risk indicator calculated according to Formula 1 or Formula 2 has a ratio between analyzing indicators of Formulae 1 and 2.

8. The apparatus of claim 6, wherein the display unit displays each of the thermal risk indicators calculated according to Formulae 1 and 2.

9. The apparatus of claim 6, wherein the display unit emphasizes and displays the thermal risk indicator calculated according to Formula 1 when the estimated value ΔT(t) reaches the possible maximum temperature rise range value ΔT_{MAX}.

10. The apparatus of claim 6, wherein the display unit emphasizes and displays the thermal risk indicator calculated according to Formula 2 when the elapsed time ‘t’ reaches the stable used time t_{SU}.

11. The apparatus of claim 9, wherein the display unit emphasizes and displays the thermal risk indicator calculated according to Formulae 1 or 2 by using at least one of bold lettering, colored letters, and flickering letters.

12. The apparatus of claim 2, further comprising a determination unit for determining whether the elapsed time ‘t’ reaches the stable used time t_{SU}.

13. The apparatus of claim 12, wherein the determination unit automatically reduces or shuts off a transmission voltage according to a determination result obtained by the determination unit.

14. The apparatus of claim 12, wherein the determination unit displays an alarm message or information data according to a determination result obtained by the determination unit.

15. The apparatus of claim 2, wherein, when a position of a biological tissue to which the ultrasound beam is irradiated is not changed and an operational state of a system is changed, the estimated value ΔT(t), the possible maximum temperature rise range value ΔT_{MAX}, and the stable used time t_{SU} are updated and the elapsed time ‘t’ continues to elapse.

16. The apparatus of claim 2, wherein, when a position of a biological tissue to which the ultrasound beam is irradiated is changed and an operational state of a system is maintained, the possible maximum temperature rise range value ΔT_{MAX} and the stable used time t_{SU} are maintained, and the estimated value ΔT(t) and the elapsed time ‘t’ is reset.

17. The apparatus of claim 2, wherein, when a type of a biological tissue to which the ultrasound beam is irradiated or an operational state of a system is changed, the possible maximum temperature rise range value ΔT_{MAX} and the stable used time t_{SU} are calculated again, and the elapsed time ‘t’ is reset.

18. The apparatus of claim 2, wherein the display unit displays the thermal risk indicator as any one of a standard image mode in which a temperature of a normal state of a biological tissue increases and a burst mode for an acoustic radiation force image, in which a temperature of a biological tissue changes remarkably.

19. A method of displaying a thermal risk indicator, the method comprising:
measuring at least one analyzing indicator about a risk of a thermal bio-effect by analyzing an ultrasound beam generated from an ultrasound output unit of a transmitting transducer;
calculating a thermal risk indicator based on the at least one analyzing indicator; and
displaying the thermal risk indicator.

20. The method of claim 19, wherein the at least one analyzing indicator comprises an estimated value ΔT(t) by which a temperature increases hourly, a possible maximum temperature rise range value ΔT_{MAX} that is calculated with respect to a preset operating condition, an elapsed time ‘t’ after the operating condition is reset, or a stable used time t_{SU} for a tissue and position to which the ultrasound beam is irradiated.

21. The method of claim 20, wherein the thermal risk indicator is calculated according to Formula 3 or Formula 4 below:
$$\begin{matrix}\frac{Δ T ( t )}{Δ {T}_{MAX}} & ( 3 ) \\ \frac{t}{{t}_{SU}} & ( 4 )\end{matrix}$$

22. The method of claim 21, wherein the thermal risk indicator calculated according to Formula 3 or Formula 4 has a ratio between analyzing indicators of Formulae 3 and 4.

23. A computer readable recording medium having recorded thereon a program for executing the method of claim 19.
